(19) United States
(12) Patent Application Publication
(10) Pub. No.: US 2022/0288254 A1
(43) Pub. Date: Sep. 15, 2022

(54) SMART, RAPID, SAFE DISINFECTION METHODS, DEVICES, AND SYSTEMS FOR FLUSHOMETERS, TOILETS, FAUCETS, BATHS, AND SHOWERS

(71) Applicant: Lumen Catheters, LLC, Marlborough, MA (US)

(72) Inventors: Adam E. M. Eltorai, Marlborough, MA (US); Charles W. Henry, Denver, CO (US)

(21) Appl. No.: 17/693,769

(22) Filed: Mar. 14, 2022

Related U.S. Application Data
(60) Provisional application No. 63/160,348, filed on Mar. 12, 2021.
(60) Provisional application No. 63/241,009, filed on Jan. 1, 0001.

(51) Int. Cl. A61L 2/10; A61L 2/24; A61L 2/00; A47K 17/00

(52) U.S. Cl. CPC A61L 2/10; A61L 2/24; A61L 2/0047; A47K 17/00; A61L 2202/11; A61L 2202/14

(57) ABSTRACT

A light-based sterilization source directs a blue light at a touched or contacted surface for eliminating harmful viral or bacterial contaminants. The blue light is outside the range of harmful UV light while delivering an effective decontaminating light source for eradication of pathogenic microorganisms. An irradiation device includes a light source disposed for irradiating a bathroom or fixture surface for sterilization. A corresponding system for sterilization of contaminated surfaces using the irradiation device includes a blue light source having a wavelength outside a harmful spectrum such as the UV (ultraviolet) spectrum.

### RELATED APPLICATIONS

This patent application claims the benefit under 35 U.S.C. § 119(e) of U.S. Provisional Patent App. No. 63/160,348, filed Mar. 12, 2021, entitled “SMART IMMEDIATE SAFE TOILET DISINFECTION METHOD, DEVICES, AND SYSTEM,” and U.S. Provisional Patent App. No. 63/241,009, filed Sep. 6, 2021, entitled “ANTIMICROBIAL LIGHT EMITTING FAUCETS AND SHOWER HEADS,” both incorporated herein by reference in entirety.

### BACKGROUND

Physical contact represents a viable transmission path for many harmful bacterial and viral contaminants. Indirect contact through intermediate surfaces can be mitigated through frequent cleaning of surfaces prone to contact from multiple people in a short time, such as handrails, doorknobs, elevator buttons, and the like. Chemical disinfectants are one effective means to keeping commonly touched surfaces free of transmittable disease, however can be labor intensive. Radiation from certain light sources may also be effective, however the radiation can also be harmful to humans, and thus imposes overhead to contain radiation.

### SUMMARY

A light-based sterilization source directs a blue light at a bathroom fixture, surface or region for eliminating harmful viral or bacterial contaminants or other pathogens. The blue light is outside the range of harmful UV light while capable delivering an effective decontaminating, antimicrobial light source for eradication of pathogenic microorganisms, including SARS CoV-2. Control and projection structures determine usage patterns and events indicative of usage and/or contact. Based on detected usage, blue light is directed from a source to an irradiated target surface for decontamination.

Transmission of pathogens has long been known to occur by touch, from person to person, and indirectly, through surfaces contaminated by a contagious person and then touched by another. Configurations herein are based, in part, on the observation that high touch surfaces may be managed by regular cleaning and/or hand sterilization by users. Substantial resources are deployed for pursuing a regular cleaning cycle and ensuring timely refilling of hand dispensers of alcohol gel. Unfortunately, conventional approaches suffer from the shortcoming that they depend on diligent execution, and still allow for periods of contagion depending on how long the pathogens remain viable on the high touch surface. Accordingly, configurations herein substantially overcome the shortcomings of conventional adherence to rigorous surface cleaning by providing an irradiating light source directed to a high touch surface for automatic, immediate sterilization and pathogen removal resulting from specific selection of the wavelength of the irradiating light. A further advantage is the benign nature of the irradiating light, being in the blue spectrum of wavelength (around 400-470 nm) that removes it from the harmful UV spectrum.

The disclosed approach employs an irradiation device including a light source disposed for irradiating a bathroom or fixture surface for sterilization. A system for sterilization of contaminated surfaces using the irradiation device includes a blue light source having a wavelength outside a harmful spectrum such as the UV (ultraviolet) spectrum.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a usage environment including bathroom fixtures having high touch surfaces suitable for use with configurations herein;

FIGS. 2A and 2B show a toilet lid activated antimicrobial light source in the environment of FIG. 1;

FIGS. 3A and 3B show a commercial fixture with an automated flushing valve integrated with an antimicrobial light source as in FIGS. 1-2B;

FIG. 4 shows a shower fixture having an antimicrobial light source as in FIGS. 1-3B; and

FIG. 5 shows a faucet fixture having an antimicrobial light source as in FIGS. 1-4.

### DETAILED DESCRIPTION

Depicted below is an example of various configurations of the antimicrobial light generation device, referred to as a “Flushometer.” Several views and arrangements are shown; other embodiments may be apparent to those of skill in the art by slight variations to the form factor and electrical circuit as shown.

FIG. 1 is a context diagram of a usage environment 10 including high touch surfaces in a bathroom, fixture or personal hygiene arrangement appropriate for use with configurations herein. Restroom environments present a myriad of high touch surfaces resulting from repeated users, particularly in public contexts. Configurations herein present a system, method and apparatus for automated illumination of high touch surfaces activated by user actions in a bathroom environment. Usage patterns incurred by bathroom fixture usage are leveraged to provide activation of antibacterial light, based on surfaces or controls that are touched or manipulated by a user through normal uses. The result is a self-sustaining pattern of regular disinfecting illumination triggered after each usage or visit.

Referring to FIG. 1, a method of rapidly disinfecting personal hygiene surfaces using antimicrobial light include disposing a light source 20 for projection onto a fixture surface 22, where the fixture surface is located in a region associated with personal hygienic care. Bathroom fixtures, such as a toilet 50 or water closet, sink faucet, and shower valve generally require users to manipulate the respective seat or faucet/valve handle. A sensor 30 is invoked for detecting a hygienic care usage associated with the fixture surface 22. The sensor 30 activates a power source or supply for activating the light source 20 for sterilizing irradiation 32 of the fixture surface 22 in response to the detected usage. Any suitable sensing medium may be employed by the sensor, such as a switch closure from interference with the toilet seat 24, or obscuring a light sensor from ambient light, for example. Hygienic care usage refers to detection of a user engaging a bathroom and using (touching and manipulating) one or more of the fixtures, typically a toilet, sink faucet or shower valve.

In the example of FIG. 1, the light source 20 is located on an underside of a toilet seat 24 for directing the irradiating light 32 around the bowl surface. The sensor 30 may be a contact closed by the toilet seat 24 for establishing a current flow, and disposed by an attachment 25 or adherence for retrofit applications. Since LED lights used for the light source 20 have low current draw, they may be powered by any suitable power source including AC, low voltage (5/12/24V) transformed DC, or battery powered (it should be noted that LED elements are generally polarized and require DC current). A timer ensures an effective duration in the event the seat 24 and/or lid 26 are left in a down, or horizontal position.

In contrast to conventional approaches, the blue light source has a wavelength between 400-470 nm, whereas ultraviolet (UV) light in conventional approaches has a wavelength around 200-300 nm. The blue light exhibits a particular effectiveness against pathogenic organisms at a wavelength substantially around 405 nm, as discussed further below. Ultraviolet (UV) light is a form of electromagnetic radiation commonly acknowledged to have a wavelength from 10 nm to 400 nm. The blue light is in the visible spectrum that exhibits only nominal, non-harmful radiation. While UV light in general may remain an effective sterilization medium, it typically requires shielding for protection from the UV radiation.

Upon invocation based on the sensed position of the toilet seat and/or lid, the light source 20 is energized for irradiating the fixture surface with a far UV light source having a wavelength selected based on eradication of surface pathogens. This effectively defines a balance between effective mitigation of pathogens using a light source safe for direct or indirect exposure to humans.

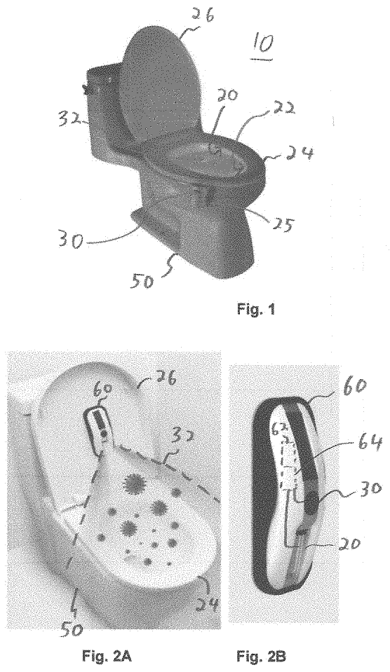

FIGS. 2A and 2B show a toilet lid activated antimicrobial light source in the environment of FIG. 1. Referring to FIGS. 1, 2A and 2B, detecting the hygienic care usage of the toilet is based on a horizontal to vertical transition of the hinged lid 26. A suitable attachment adheres a containment 60 including the light source 20 to the surface of the hinged lid 26. The sensor 30 may be an optical sensor obscured from ambient light when the lid 26 is down (horizontal). Alternatively, light activation may be based on receiving a gravitational based switch input indicative of the horizontal to vertical transition of the lid 26. Upon detection of the lid 26 in the down (horizontal position), the light source 30 activates based on the switch input. Similarly, in the case of optical sensing, the hinged lid 26 surface obscures an optical sensor in at least one of a horizontal or vertical position, and detection of hygienic care usage is based on a signal from the optical sensor, meaning that the horizontal position shield the sensor 30 from ambient light.

Upon detection of usage, the light source activates for a duration based on an expected use of the region for hygienic care and a time sufficient to irradiate the fixture surface. In other words, an affirmative user action to commence a hygienic care usage occurs at the beginning of use. A duration of irradiation extends for a reasonable duration of usage, followed by sufficient time to mitigate, sterilize and/or kill pathogens. Alternatively duration may distinguish the end of termination for bathroom use, such as turning off a faucet. However, as the light source 20 draws little energy, activating the light source 20 during the usage period, as opposed to only following the usage period, does not achieve substantial efficiency gains. A typical irradiation cycle can eliminate about 90% of pathogens in about 10 min., 95% around 30 min., and about 99.9% after an hour of irradiation.

A power supply 62 connects to the light source 20 via an activation circuit 64 for activating the light source 20 in response to the detected hygienic care usage upon input from the sensor 30. Batteries are sufficient for powering an LED element or array used for the light source 20, however alternate power and light fixtures may be employed.

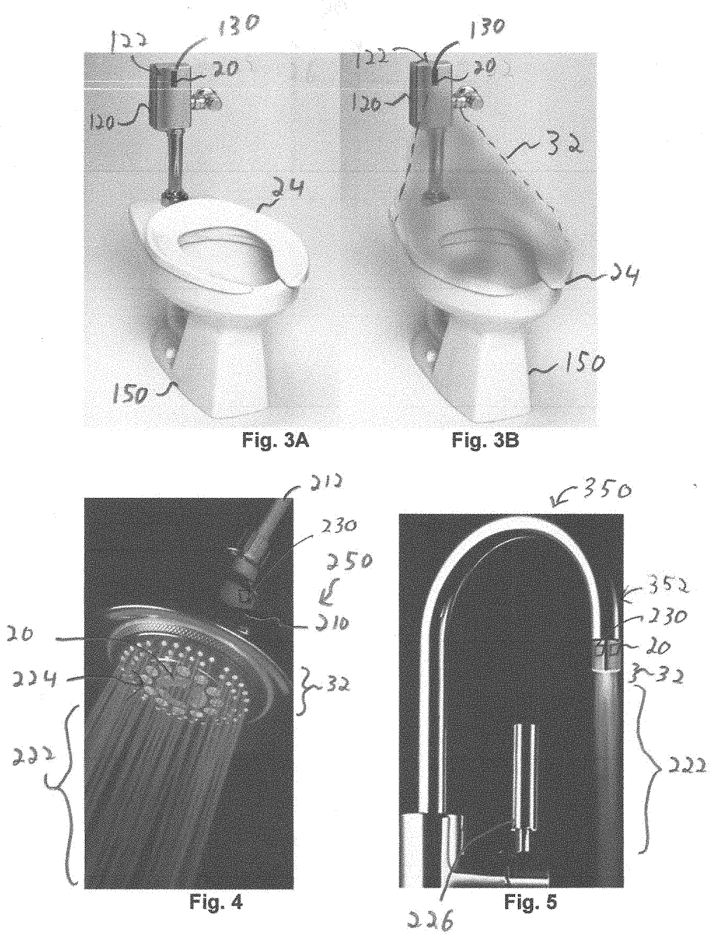

FIGS. 3A and 3B show a commercial fixture with an automated flushing valve integrated with an antimicrobial light source as in FIGS. 1-2B. Referring to FIGS. 1, 3A and 3B, commercial toilet fixtures 150 generally omit a storage tank and instead rely on a shorter but greater pressure and volume of a plumbed water source. Commercial fixtures often employ a motion sensor or optical sensor flush valve 120 that operate a flush operation upon detecting a presence of a user followed by a departure, and conclude that the hygienic care usage has concluded. Detection of hygienic care usage is based on an interface with a motion sensor valve control operative to activate a flushing discharge. Commencement of irradiation by the light source 20 is received with the activation of the optical sensor flush valve 120, and applies irradiating light 32 in response. Automated flush valves are often electric (typically battery), and the activation of the light source 20 is based on the same electrical signal used for flushing and irradiates through the same window 122 used for user detection.

FIG. 4 shows a shower fixture having an antimicrobial light source as in FIGS. 1-3B. Referring to FIGS. 1 and 4, a shower fixture 250 commences a water flow 222 for hygiene care usage. In FIG. 4, detection of hygienic care usage is based on a flow rate of liquid (tap water) through a discharge vessel 210, such as a shower head.

In FIG. 4, a system for rapidly disinfecting shower surfaces invokes antimicrobial light including the light source 20 for projection onto a fixture surface located in a region associated with personal hygienic care. A shower stall or enclosure receives the irradiated light 32. A sensory input detects a hygienic care usage associated with the fixture surface, from a flow sensor 230 in the shower fixture 250 or associated water flow such as a supply pipe 212. The flow sensor 230 initiates an activator to power the light source 20 for sterilizing irradiation of the fixture surface in response to the detected usage. The light source 20 is a hermetically sealed (waterproof) LED element on a discharge surface 224 from which the water emanates, or optionally just behind and projected through the water stream.

FIG. 5 shows a faucet fixture having an antimicrobial light source as in FIGS. 1-4. Operation occurs to irradiate the user's hands during washing, and also a sink surface receiving the flowing water. Referring to FIGS. 1 and 5, the method of rapidly disinfecting the hands of users of faucets invokes antimicrobial light integrated into the faucet 350. This includes disposing a light source 20 for projection onto a fluid discharge stream from a discharge vessel 352 and directed into a region associated with personal hygienic care. The sensor 30 detects a fluidic flow through the discharge vessel 352 and activates the light source 20 for sterilizing irradiation of the fluid discharge stream and appurtenant areas in response to the detected fluidic flow.

The light source 20 receives an input from a flow sensor 230 on the discharge vessel for detecting the fluidic flow 222, and activates the light source 20 using any suitable power source as disclosed above. The fixture 350 disposes the light source 20 within the discharge vessel 352 adjacent a termination and discharge end of the discharge vessel, and activates the light source towards the discharge end from an interior of the discharge vessel 352 for irradiating the immediate area, including a sink surface and hands of a user. The flow sensor 20 is shown in communication with an interior of the discharge vessel 352 for detecting the water flow 222, however could be disposed alternately for detecting usage, such as in a supply line or detecting touch or position of a handle 226 for operating the fixture 350.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of rapidly disinfecting toilet, sink, bath, and shower surfaces using antimicrobial light, comprising:
disposing an antimicrobial light source for projection onto a surface;
detecting toilet, sink, bath, or shower usage or human proximity; and
activating the light for disinfecting irradiation of the surface in the absence of detected usage or human proximity and deactivating the light with detected usage or human proximity.

2. The method of claim 1 wherein detecting the usage is based on sensing at least one of motion, pressure, flow, time, light, sound, temperature, and chemical change.

3. The method of claim 1 further comprising:
sufficiently activating the light source to provide surface disinfection.

4. A system for rapidly disinfecting toilet, sink, bath, and shower surfaces using antimicrobial light, comprising:
an antimicrobial light source for projection onto a surface;
a sensor for detecting toilet, sink, bath, or shower usage or human proximity; and
a light activator and deactivator.

5. The system of claim 4 wherein the light further comprises far UVC wavelength.

6. The system of claim 4 wherein the light further comprises at least one of a UV or non-UV wavelength.

7. The system of claim 4 wherein the light source is integrated into or attached onto the flushometer, toilet, faucet, or shower head.

8. A method of rapidly disinfecting the hands of users of faucets using safe, antimicrobial light, comprising:
disposing an antimicrobial light source for targeted projection onto the hands of the users;
detecting faucet usage; and
activating the light source for disinfecting irradiation of the user hands in response to detected faucet usage and deactivating the light with absence of detected usage.

9. The method of claim 8 wherein detecting the usage is based on sensing at least one of motion, pressure, flow, time, light, temperature, sound, and chemical change.

10. The method of claim 8 wherein the light source is integrated into or attached onto the faucet.

11. A system for rapidly disinfecting the hands of users of faucets using safe, antimicrobial light, comprising:
an antimicrobial light source for targeted projection onto the hands of the users;
a sensor for detecting faucet usage; and
a light activator and deactivator.

12. The system of claim 11 wherein the light further comprises far UVC wavelength.

13. The system of claim 11 wherein the light comprises at least one of a UV or non-UV wavelength.

14. The system of claim 11 wherein the light source is integrated into or attached onto the faucet.

15. A method of rapidly disinfecting users of baths or showers using safe, antimicrobial light, comprising:
disposing an antimicrobial light source for projection onto the users;
detecting bath or shower usage; and
activating the light source for disinfecting irradiation of the users in response to detected bath or shower usage and deactivating the light with absence of detected usage.

16. The method of claim 15 wherein detecting the usage is based on sensing at least one of motion, pressure, flow, sound, time, light, temperature, and chemical change.

17. The method of claim 15 wherein the light source is integrated into or attached onto the bath faucet or shower head.

18. A system for rapidly disinfecting users of baths or showers using safe, antimicrobial light, comprising:
an antimicrobial light source for projection onto the users;
a sensor for detecting bath or shower usage; and
a light activator and deactivator.

19. The system of claim 18 wherein the light further comprises far UVC wavelength.

20. The system of claim 18 wherein the light comprises at least one of a UV or non-UV wavelength.

21. The system of claim 18 wherein the light source is integrated into or attached onto the bath faucet or shower head.

22. A method of rapidly disinfecting toilet surfaces using antimicrobial light, comprising:
disposing an antimicrobial light source from a flushometer for projection onto toilet surfaces;
detecting toilet usage or human proximity; and
activating the light for disinfecting irradiation of the surface in the absence of detected usage or human proximity and deactivating the light with detected usage or human proximity.

23. The method of claim 22 wherein detecting the usage or user proximity is based on sensing at least one of motion, pressure, flow, time, light, sound, temperature, and chemical change.

24. The method of claim 1 further comprising: sufficiently activating the flushometer light source to provide toilet surface disinfection.

25. A system for rapidly disinfecting toilet surfaces using antimicrobial light, comprising:
an antimicrobial light source from a flushometer for targeted projection onto toilet surfaces;
a sensor for detecting toilet usage or human proximity; and
a light activator and deactivator.

26. The system of claim 25 wherein the light further comprises far UVC wavelength.

27. The system of claim 25 wherein the light further comprises at least one of a UV or non-UV wavelength.

28. The system of claim 25 wherein the light source is integrated into or attached onto the flushometer.
